(19) United States
(12) Patent Application Publication
(10) Pub. No.: US 2012/0157854 A1
(43) Pub. Date: Jun. 21, 2012

(54) SYSTEM AND METHOD FOR GAINING PERCUTANEOUS ACCESS TO A BODY LUMEN

(75) Inventors: Michael R. Kurrus, Ellettsville, IN (US); Michael E. Arnold, Ellettsville, IN (US)

(73) Assignee: Cook Incorporated, Bloomington, IN (US)

(21) Appl. No.: 12/969,003

(22) Filed: Dec. 15, 2010

(51) Int. Cl. A61M 25/06; A61B 8/00; A61M 29/00

(52) U.S. Cl. 600/461; 604/164.01; 604/164.03

(57) ABSTRACT

Systems and methods for gaining percutaneous access to a body lumen, preferably without a guidewire, are provided herein. The system can include a tubular medical device and a needle with a distal tip. A side port is provided through a wall of the tubular medical device, to be in communication with a distal end opening of the device via a passageway. The needle can be positioned within the side port to extend the distal tip beyond the distal end opening. In this position, the distal tip can form an entry opening in the body lumen, and a distal portion of the device can be advanced over the indwelling needle into the entry opening. The needle can then be withdrawn from the tubular medical device, while maintaining a portion of the tubular medical device in place within the body lumen.

### TECHNICAL FIELD

The present disclosure relates to systems and methods for gaining percutaneous access to a body lumen, such as an artery or vein, particularly with at least a tubular medical device and a needle.

### BACKGROUND

Many medical procedures require percutaneous placement of an interventional medical device, such as a catheter, into a body lumen such as an artery or vein. Such interventional medical devices may be used for, among other things, blood pressure monitoring, blood sampling, and administering fluids and medicaments to a patient. In one aspect, percutaneous access to a patient's central venous system is an important aspect of administering intravenous therapy. It is desirable that the therapy be administered in the superior vena cava of the central venous system. In order to gain access, introducer devices are commonly used, through which other medical devices, such as a central venous catheter (CVC), are inserted. One such example of a CVC is a peripherally inserted central catheter (PICC). A PICC typically has one or more tubes, which are externally accessible by a clinician, that converge into a single catheter body that is internally implanted in a vein of the patient. The tubes are adapted to receive medicaments, which are then released through a distal tip of the catheter body into the central venous system of the patient.

The most common technique used by the clinician to gain percutaneous access to a central venous system of a patient with a PICC is a standard or modified Seldinger technique. This technique involves the clinician first inserting a needle through the patient's skin at a peripheral location and into a vein to form a venotomy. The clinician then inserts the guidewire through the passageway of the needle and into the vein. A distal end of the guidewire may then be translated to the target site. Next, the clinician removes the needle from a proximal end of the guidewire, and following removal of the needle, the clinician inserts an introducer sheath and dilator assembly over the proximal end of the guidewire and into the vein. The introducer sheath and dilator assembly typically includes a splittable introducer sheath and a dilator to facilitate the ease of insertion and creation of a larger opening in the venotomy. The clinician then removes the dilator, and may even subsequently remove the guidewire from the lumen of the introducer sheath, leaving the introducer sheath inserted in the body. The clinician then usually places the PICC catheter within the lumen of the introducer sheath and translates the catheter tip to the target site. Thereafter, the introducer sheath is removed from the body.

There are many disadvantages with the conventional method described above. Many clinicians are generally unskilled in the Seldinger technique of placing a guidewire within an access needle and placing a sheath over a guidewire that has been introduced to the body. The conventional Seldinger technique requires complex preparation and handling of many instruments. In turn, the procedure requires a large sterile field for the prevention of contamination and introduction of infection during insertion and removal of the many instruments.

Moreover, when using the conventional Seldinger technique, it is difficult to stabilize the needle with one hand at a suitable angle relative to the body lumen while skillfully inserting the guidewire having a diameter of about 0.014 to 0.038 inches (0.37 to 0.97 mm) through the needle lumen having an internal diameter of about 0.042 inches (1.07 mm) (18 gauge needle) or about 0.0155 inches (0.14 mm) (22 gauge needle) with the other hand. During this part of the procedure, the clinician can inadvertently cause the needle to be withdrawn or “pop out” from the body lumen. Furthermore, to avoid blood occlusion, insertion of the guidewire must be quickly and sterilely performed right after the needle is inserted as blood will flash or enter into the needle lumen. The blood consequently will begin to clot immediately within 1 to 2 minutes after needle insertion, thereby occluding the needle lumen to a degree, which can prevent the guidewire from being fully inserted through the needle lumen. When this occurs, the clinician must either flush the needle lumen with saline while maintaining the percutaneous access to the body lumen or remove the occluded needle so that a nonoccluded needle can be inserted. As can be imagined, the additional time required as a consequence of the potential complications from use of a guidewire in the conventional Seldinger technique can lead to further adverse complications. Especially in an emergency or intensive care setting, the total time to gain percutaneous access in order to implant an interventional medical device can be critical to a successful outcome in an emergency procedure.

Another one of the disadvantages of the conventional Seldinger technique is the use of larger gauge needles. Large gauge needles are required in order to accommodate guidewires that are sized to be sufficiently rigid to properly support and lead many standard catheters and other interventional medical devices commonly used in medical procedures. For example, such a guidewire is typically constructed to have an outer diameter in a range on the order of about 0.035 to 0.038 inches (0.89 to 0.97 mm). As a result, needle lumens to accommodate such sized guidewires require needles that are at least 18-gauge or larger. However, the outer diameter of an 18-gauge needle (about 0.050 inches (1.27 mm) is just large enough to cause damage to the lumen or cause excessive bleeding when it does not enter the lumen correctly, or when it inadvertently penetrates an organ or other unintended body structure.

Thus, what is needed is a system for gaining percutaneous access to a body lumen that avoids the complications associated with guidewire insertion within a needle. Further, what is needed is a method for gaining percutaneous access to a body lumen, such as the Seldinger technique that is further modified so that initial percutaneous access to the body lumen is obtainable without use of a guidewire. It would be further desirable to use a smaller gauge needle and even more desirable to gain percutaneous access to a body lumen in fewer steps.

### BRIEF SUMMARY

A system and method for gaining percutaneous access to a body lumen including a tubular medical device and a needle with a distal tip are provided. In one system example, the needle can have a distal tip configured to create an entry opening in the body lumen. The tubular medical device can have a proximal end, a distal end opening, and a distal portion. A side port may be formed within the wall of the tubular medical device, such as the distal portion thereof. The side port can be in communication with the distal end opening through a passageway that extends at least between the side port and the distal end opening. The side port, the passageway, and the distal end opening can be configured to receive the needle so that the distal tip of the needle is extendable beyond the distal end opening of the tubular medical device prior to creation of the entry opening with the distal tip of the needle. The passageway and/or the needle may have features to facilitate slidability and/or sealability between the side port, the passageway, and the distal end opening and the needle.

In one aspect, the tubular medical device can include a combination of an introducer sheath and a dilator inserted within the introducer sheath. A portion of the dilator can have the side port. The dilator can be removed from the introducer sheath after the introducer sheath has gained access to the body lumen. The introducer sheath provides a conduit for subsequent introduction of a catheter, such as a central venous catheter or PICC. In another aspect, the tubular medical device is the catheter, instead of the assembly of the introducer sheath and dilator, where the catheter includes the side port.

In another system example, a tubular medical device can have a proximal end and a distal end opening, a tapered distal portion to dilate the entry opening of the body lumen, and a side port formed through a wall of the tapered distal portion. The needle can reside within the side port, the passageway, and the distal end opening so that the distal tip is extended beyond the distal end opening of the tubular medical device for creation of the entry opening. A length of the tapered distal portion can be translatable over the needle into the entry opening. The needle can be removable from the side port, the passageway, and the distal end opening subsequent to introduction of the tapered distal portion of the tubular medical device into the body lumen through the access opening.

In another embodiment, a method for gaining percutaneous access to a body lumen is provided. The method can include at least one of the following steps. A tubular medical device can be provided, which can have a proximal end, a distal end opening, a distal portion, a side port formed in the distal portion, and a passageway in communication with at least the distal opening end and the side port. A needle can be provided having a distal tip configured to create an entry opening in the body lumen. The distal tip of the needle can be extended through the side port and translated along the passageway to exit beyond the distal end opening of the tubular medical device. An entry opening can be formed in the body lumen with the distal tip of the needle, while the needle resides within the side port, passageway, and distal end opening. The distal portion of the tubular medical device can be advanced over the indwelling needle into the entry opening so that the tubular medical device gains percutaneous access to the body lumen. This step may be performed without using a guidewire. The needle may be withdrawn from the side port of the tubular medical device, while a portion of the tubular medical device is maintained in place within the body lumen.

### BRIEF DESCRIPTION OF THE DRAWINGS

FIG. 1 is a side elevation view of a system for gaining percutaneous access to a body lumen of a patient, the system including an access needle extending through a side port in a tubular medical device.

FIG. 2 is a side elevation view of a first embodiment of a tubular medical device.

FIG. 3 is an enlarged side view of a distal end of a tubular medical device.

FIGS. 4A-4D are various cross-sectional views of a distal portion of a tubular medical device.

FIG. 5 is a side elevation view of a second embodiment of a tubular medical device.

FIGS. 6A-6E are side elevation views, depicting various steps of a method for gaining percutaneous access to a body lumen of a patient.

FIG. 7 is a perspective view of a needle being used in combination with an ultrasound transducer probe.

FIG. 7A is a side elevation view, depicting an alternative step of a method for gaining percutaneous access to a body lumen of a patient, using the ultrasound transducer probe in FIG. 7.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same.

In the following discussion, the terms “proximal” and “distal” will be used to describe the opposing axial ends of inventive devices, as well as the axial ends of various component features. The term “proximal” is used in its conventional sense to refer to the end of the device (or component thereof) that is closest to the clinician during use of the device. The term “distal” is used in its conventional sense to refer to the end of the device (or component thereof) that is initially inserted into the patient, or that is closest to the patient during use.

The inventive percutaneous access system may be used, among other possible uses, to gain rapid access to a body lumen, such as an artery or vein, without the necessity of using a guidewire. Further, the system can include a combination of a tubular medical device and a needle, with the combination capable of at least one of forming an opening in a body lumen and dilating the body lumen opening for insertion of a larger medical device to gain access therein. The tubular medical device can include an introducer sheath and dilator assembly. When a dilator is used as a component of the assembly, it can be withdrawn from the introducer sheath and an interventional medical device such as the intravascular catheter can be inserted within the introducer sheath. Alternatively, the tubular medical device can include just the interventional medical device such as an intravascular catheter with a tapered tip, without a dilator, to gain rapid percutaneous access to a body lumen. Non-limiting examples of medical procedures include the introduction or removal of fluids and/or solids from a body lumen of a patient.

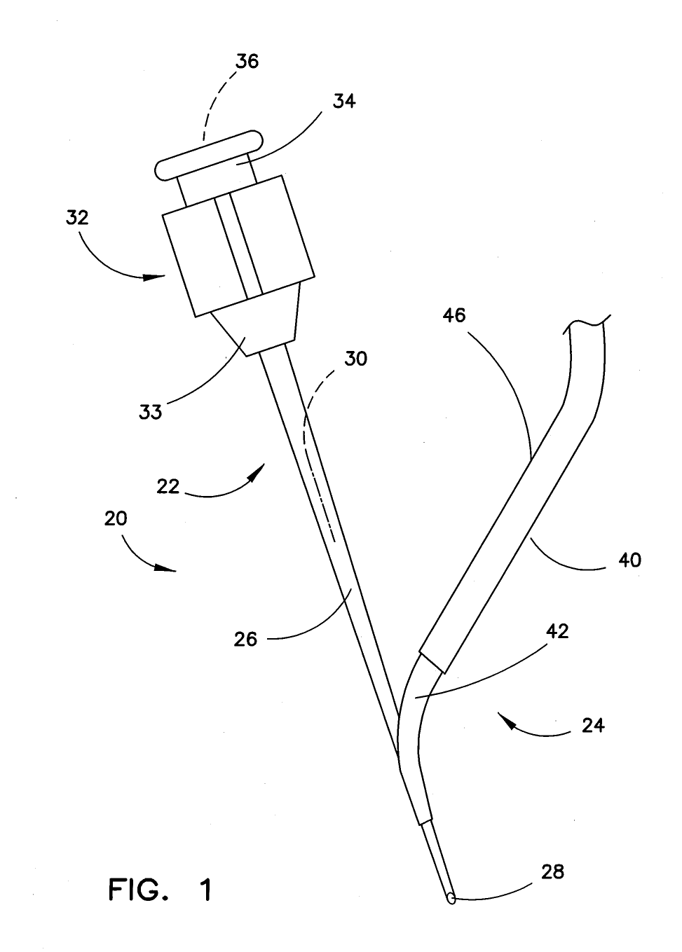

FIG. 1 illustrates one embodiment of a percutaneous access system 20 including a needle 22 extending through a portion of a tubular medical device 24. Needle 22 can be a cannula 26 of any material, preferably biocompatible such as stainless steel, having a lancet beveled tip 28 (shown enlarged) and a lumen 30 extending therethrough, although needle 22 can also be a solid needle such as a trocar tip that does not include a lumen. Needle 22 can be any conventional size, but is preferably a smaller gauge needle, such as a 21-gauge needle or smaller, typically having an outer diameter up to 0.032 inch (0.81 mm) and an inner diameter up to 0.022 inch (0.56 mm). A hub 32 can be attached at an end 33 of cannula 26, opposite tip 28. Hub 32 can be plastic and can be equipped with a fluid coupling 34, such as a standard Luer lock type connector. Hub 32 can have a passageway 36 extending therethrough in alignment with and in fluid communication with lumen 30. Passageway 36 can be at least as large in diameter as lumen 30. Needle 22 can be any selected length as is appropriate for the particular body lumen which is to be catheterized.

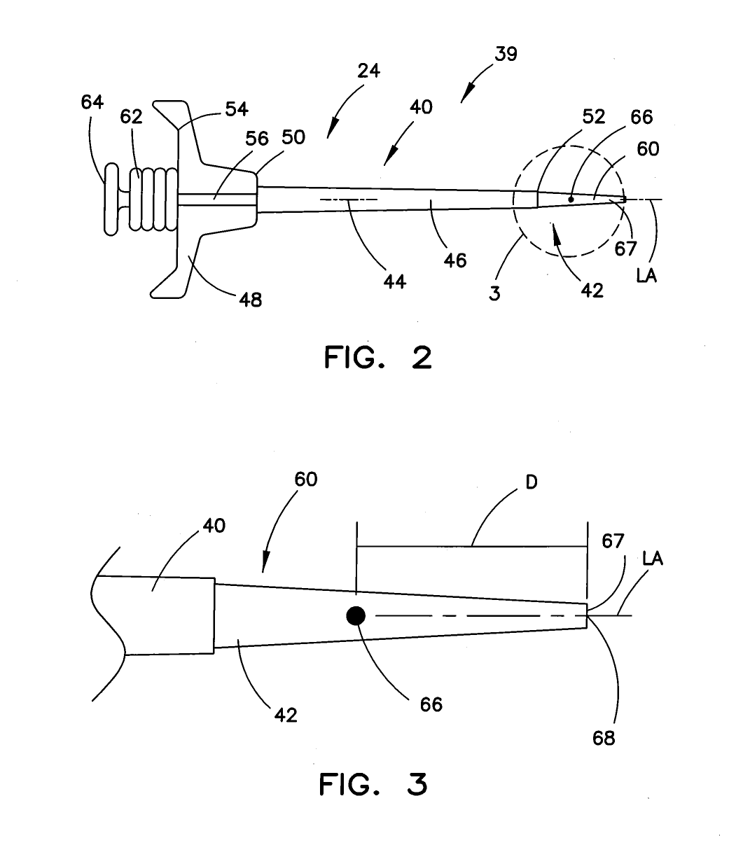

With further reference to FIG. 2, tubular medical device 24 can include an assembly 39 of an introducer sheath 40 and a dilator 42. Tubular medical device 24 may be adapted to provide external percutaneous access to a body lumen. System 20 can be utilized without the use of a guidewire, although a guidewire can be used if desired. Instead, tubular medical device 24 is configured to be inserted over needle 22, while the needle is in place in a body lumen as explained below in further detail. Dilator 42 is shown inserted completely through an access lumen 44 of introducer sheath 40 and preferably locked to the introducer sheath to form assembly 39.

Introducer sheath 40 can include a sheath body 46 extending from a handle 48. Sheath body 46 is a tubular body having a proximal end 50 and a distal end 52, with a passageway extending longitudinally therethrough about a longitudinal axis LA. Sheath body 46 can be made of low friction biocompatible polymers or fabrics. Non-limiting examples of a suitable material for the sheath body includes polytetrafluoroethylene (PTFE), ePTFE, polyethylene terephthalate (PET), polyamides such as nylon, polyether amide (PEBA), polyurethanes, or the like. Dilator 42 may be of any conventional composition. One particularly preferred composition is a lubricous fluoropolymer composition, such as PTFE. However, a significant difference between a conventional dilator, such as that commonly used in combination with the introducer sheath and the dilator of the system described herein is that the dilator is provided with a side port as will be further discussed below.

Handle 48 can be attached to proximal end 50 of sheath body 46. An opening can extend through a central portion of handle 48 along longitudinal axis LA, with the handle opening in fluid communication with the passageway of sheath body 46 to define together the entire access lumen 44 of introducer sheath 40. One or more tabs 54, preferably disposed on opposite sides of access lumen 44, and extending radially outward away from longitudinal axis LA, can also be provided on handle 48. Tabs 54 can provide support for the fingers and/or thumb of the clinician and enhanced control and handling of the device during its use. Handle 48 can be formed from any rigid or semi-rigid material having sufficient structural strength for the purposes described herein. Preferably, handle 48 is insert-molded over proximal end 50 of sheath body 46. Handle 48 preferably has a weakened region 56 in a longitudinal direction, so that introducer sheath 40 is capable of being longitudinally split for removal from the patient's body. Weakened region 56 may facilitate the splitting of the handle and/or at least a portion of sheath body 46 as well. In other words, when tabs 54 are pulled radially apart from one another and downward, handle 48 and at least a portion of sheath body 46 can be readily removed by peeling introducer sheath 40 away from a device that is inserted in its access lumen. Weakened region 56 can include a pair of axial slots, one of which is shown in FIG. 2, to create a region of relatively thin material. The weakened region may further include a longitudinal region of softer material, a series of perforations, or other structural features to facilitate removing of the introducer sheath as known in the art. The tabs can be a variety of shapes and can even include surface irregularities to improve its gripping when being manipulated and pulled apart.

Dilator 42 can have an elongated body, with a substantial portion having an outer diameter closely approximating the inner diameter of access lumen 44 of introducer sheath 40. A distal end 60 of dilator 42 is shown extending beyond distal end 52 of sheath body 46. A portion of distal end 60 can be tapered to an even smaller outer cross-sectional area, e.g., the size of the needle, in order to facilitate insertion of introducer sheath 40 into a body lumen. Dilator 42 may also have a connector 62, e.g., a threaded connector, which is capable of being removably attached to a portion of handle 48 of introducer sheath 40. In addition, dilator 54 may also include a fluid coupling 64, such as a standard Luer lock type connector, at its proximal end for attachment to devices used for flushing the system, and may even include an injection cap (not shown) that is placed over the proximal end of the dilator to sealably close the proximal end.

According to the embodiment in FIGS. 2 and 3, a portion of dilator 42 is configured to receive needle 22 before insertion of the needle in a body lumen. In particular, dilator 42 can include a side port 66 through the dilator wall and an opening formed through its distal end 67. A lumen 68 can extend through at least a portion of dilator 42 fluidly connecting side port 66 with distal end opening 67. Needle 22 can be positioned through side port 66, along lumen 68, and extended distally beyond distal end opening 67.

Dilator 42 may even include an element for guiding tip 28 of needle 22 though dilator lumen 68 and side port 66 to the exterior of the system. Preferably, the guiding element comprises a plug-like structure, such as a ramp. Those skilled in the art will appreciate that alternate structures may be readily substituted for a ramp. When present, the ramp facilitates passage of the needle from dilator lumen 68 through the distal end opening to the exterior of the system. One such ramp can be found in U.S. Pat. Publ. 2007/0185521A1 to Bui et al., which is incorporated herein by reference in its entirety. In addition, side port 66 is shown extending perpendicularly through the dilator wall, but may also extend obliquely through the wall, preferably extending toward the distal end, to facilitate passage of the needle toward the distal end opening. In order to avoid puncturing the dilator wall opposite the side port, distal tip 28 of needle 22 can include a removable cap (not shown) with a blunt end that can be used during insertion of the distal tip through the dilator side port. Additional material or coating may line the dilator wall opposite the side port to strengthen and reinforce the wall against such puncturing.

According to FIG. 3, side port 66 is positioned from the distal end opening 67 a distance “D” that is sufficient to inhibit leakage of the fluid external to the patient when needle 22 is removed therefrom. In other words, when side port 66 is positioned too far from the distal end opening 67, side port 66 consequently is located external to the skin of the patient during the procedure, leading to possibly bodily fluid leakage. However, when side port 66 is positioned too close to the distal end opening 67, the material is reduced therebetween to a size where it can become susceptible to tearing. Preferably, distance D is less than the depth of the body lumen from the skin, which the depth can be measured with use of an ultrasound transducer probe as can be appreciated by one skilled in the art. For example, when balancing these criteria for most applications, distance D can be a distance in the range of about 10-20 mm. Side port 66, distal end opening 67, and lumen 68 can be sized to receive needle 22, preferably being approximately the same size as the needle. However, at least one of side port 66, distal end opening 67, and lumen 68 may be sized slightly less than the size of needle 22 (e.g., 0.1 mm less in diameter) in order for the edge that defines these features to from a seal around the needle, but sufficient to allow passage of the needle. In other examples, it may be more desirable for at least one of side port 66, distal end opening 67, and lumen 68 to be sized larger than the size of needle 22 (e.g., 0.1-0.25 mm more in diameter) to facilitate slidability of the needle through these features. The side port 66 can have the same cross-sectional area as the lumen 68.

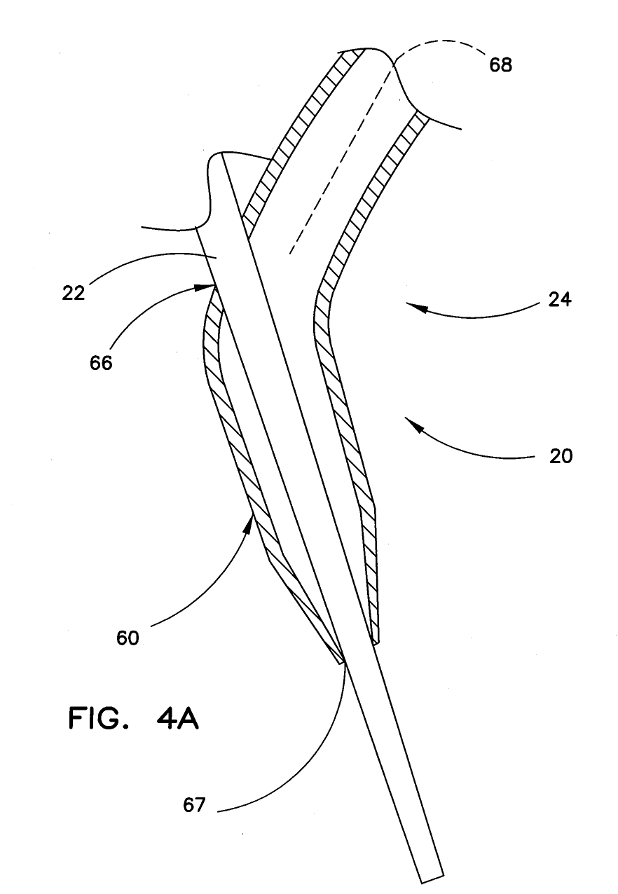

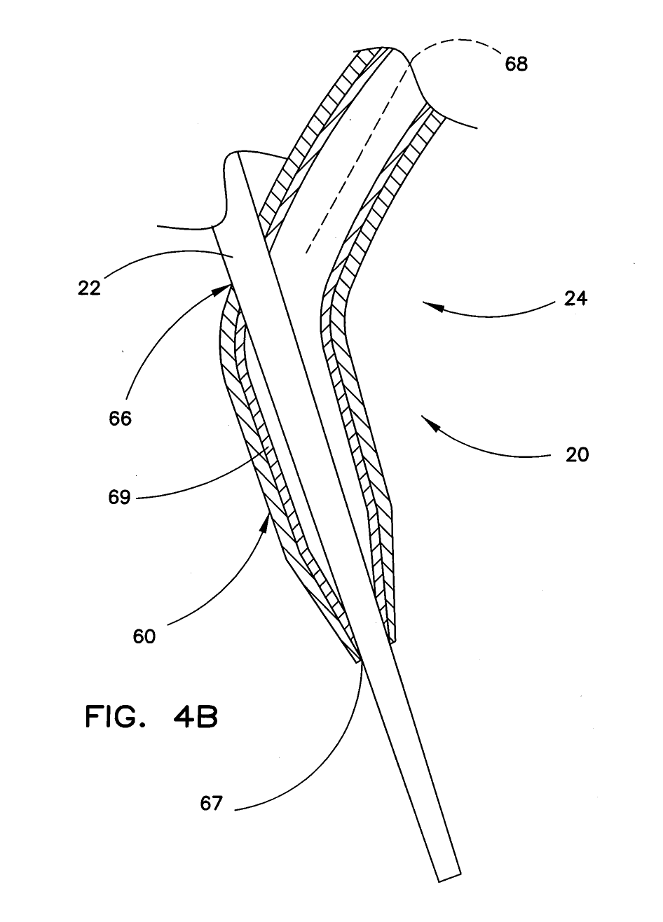

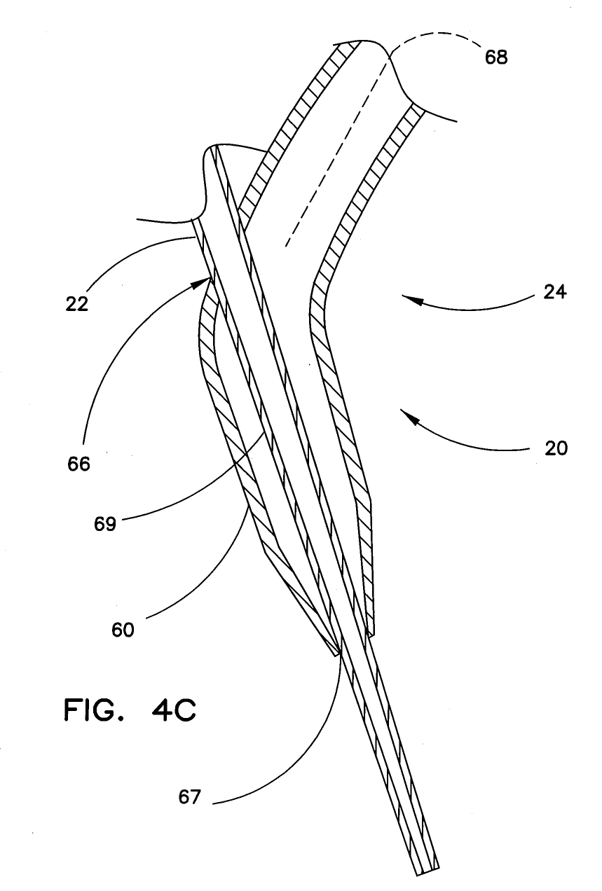

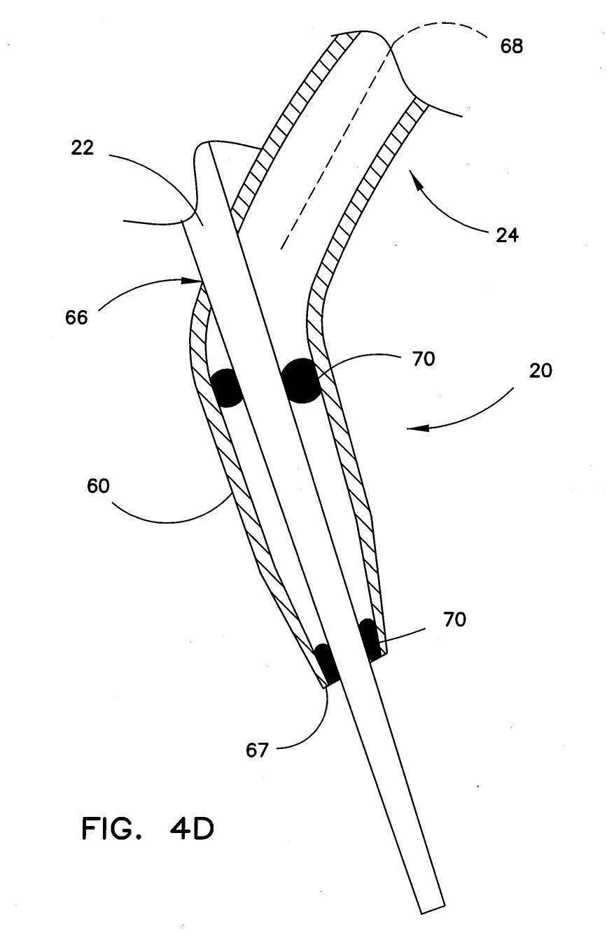

FIGS. 4A-4D illustrate various cross-sectional enlarged views of the distal end of percutaneous access system 20, having various structural features to facilitate the relative motion between needle 22 and tubular medical device 24 to be as smooth as possible. Here, needle 22 extends through side port 66, distal end opening 67, and a portion of lumen 68 therebetween of tubular medical device 24. The wall of tubular medical device 24 may have a constant wall thickness. However, it may be desirable to vary the wall thickness to become increasingly smaller toward distal end opening 67. To this end, the outer cross-sectional area of tubular medical device 24 at distal end 67 is preferably sized as close as possible to the outer cross-sectional area of needle 22 to form a smooth transition from needle 22 to tubular medical device 24. This dimensional arrangement, shown for example in FIG. 4A, can further facilitate insertion of tubular medical device 24 into a body lumen. In addition, a tapering distal end of the tubular medical device can facilitate the widening of the access opening of a body lumen when receiving the tubular medical device.

According to FIG. 4B, tubular medical device 24 may also have a lubricious coating 69, such as polytetrafluoroethylene (PTFE), silicone, or a hydrophilic coating, applied interiorly of distal end portion 60. For example, lubricious coating 69 can be applied to a portion of the luminal walls of tubular medical device 24, preferably at least a portion between side port 66 and distal end opening 67. In an alternative embodiment shown in FIG. 4C, needle 22 may also have lubricious coating 69 applied along its exterior surface. For example, lubricious coating 69 can be applied to a portion of the exterior surface of needle 22, preferably at least a portion that interacts with side port 66 and distal end opening 67 of tubular medical device 24. It can be appreciated by one skilled in the art that lubricious coating 69 can be applied to any portion of both tubular medical device 24 and needle 22, as well as to the outside surface of the tubular medical device to increase lubricity of such component. Lubricious coating 69 can facilitate the relative movement of needle 22 and tubular medical device 24 when gaining access to a body lumen. Examples of lubricous coatings and application thereof to medical devices can be found in U.S. Pat. No. 7,687,144 to Clark et al., which is incorporated herein by reference in its entirety. It will be understood that the lubricious coating can be applied in manner such as dipping, co-extrusion, heat shrinkable tubing, and the like.

FIG. 4D illustrates that one or more annular protrusions 70 can be formed along the inner surface of the luminal walls of tubular medical device 24, preferably at least along a portion between side port 66 and distal end opening 67. Here, one annular protrusion is shown proximate distal end 67 and another is shown proximate side port 67. Annular protrusion 70 is configured to reduce the amount of surface contact between the needle and the luminal walls of the tubular medical device. Annular protrusion 70 may also be sized to provide enough surface contact around the needle to form a seal, thereby reducing leakage of fluid through the side port. It can be appreciated by one skilled in the art that any combination of features in FIGS. 4B-4D can be utilized to improve the relative movement of the needle and the tubular medical device.

Access lumen 44 of introducer sheath 40 is adapted to allow the passage of other interventional medical devices, such as a blunt end or non-tipped intravascular catheter (not shown) that does not have a tip adapted to puncture the skin and/or is not adapted to access the lumen on its own. Assembly 39 with introducer sheath 40 and dilator 42 can be well suited for providing percutaneous access for such non-tipped catheters.

One example of a suitable non-tipped catheter is the SPECTRUM® Turbo-JeCT™ PICC, which is also available at Cook Incorporated of Bloomington, Ind. A PICC is a peripherally inserted central catheter that is typically associated with a central venous catheter (CVC) to be inserted in the central venous system. The PICC can include an elongated single body extending distally from a hub assembly. The single catheter body can have a proximal end and a distal tip, and one or more lumens extending therethrough. The catheter body can be made of medical grade polymers, such as polyurethane or silicone. The hub assembly of PICC can include a first port where the proximal end of the catheter body attaches thereto, and one or more second ports. Extending proximally from each second port is a tubular member extending from a proximal end and to a distal end. The hub assembly can be a structure that provides a fluid transition from one or more discrete tubular members (e.g., 1, 2, 3, 4, 5 or more tubular members) at one end to typically a corresponding number of separate, noncommunicating lumens (e.g., 1, 2, 3, 4, 5, or more lumens) that are located within the single catheter body. A fluid coupling, such as a standard Luer lock type connector, can be attached to the proximal end of each tubular member. Further, a clamping device can be disposed around each of tubular members, which is capable of shutting off fluid flow through the tubular members and preventing air aspiration due to inadvertent hub dislodgement. In addition, one or more coatings may be associated with PICC, such as a hydrophilic coating, a lubricous coating, and/or a drug coating. The coating can be associated with the catheter body by any suitable manner known in the art, such as dipping, spraying, impregnation, and the like. Drug coatings associated with such catheters are well known in the art, for example, the drug coating can be an antimicrobial useful in treating catheter-related blood stream infections, such as methicillin-resistant Staphylococcus aureus (MRSA), VARS, and vancomycin-resistant enterococcus (VRE). One such drug coating that can be beneficial in inhibiting the blood infections can comprise at least one of minocycline and rifampin, or a combination thereof.

The introducer sheath can be non-splittable as known in the art. One example of such introducer sheath is the FLEXOR@ introducer, available from Cook Incorporated of Bloomington, Ind. The FLEXOR@ introducer comprises an inner layer of a fluorocarbon, such as PTFE, a coil reinforcement, and an outer layer formed of a polymer, such as nylon. The outer layer can be bonded to the inner layer through the turns of the coil. The FLEXOR® sheath may further comprise a plurality of segments of different durometer, ranging from a higher durometer proximal segment to a lower durometer distal segment.

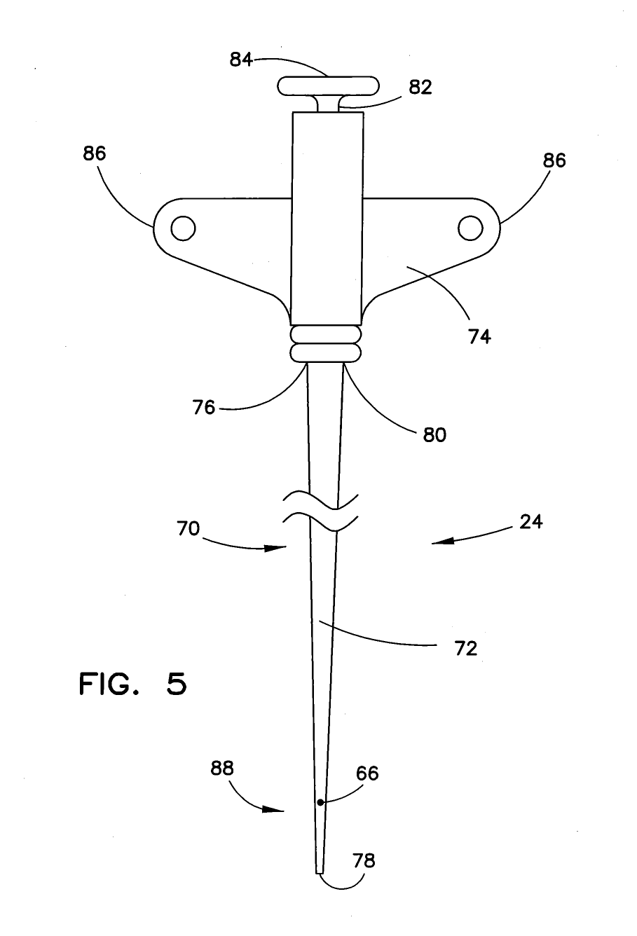

Alternatively, some intravascular catheters have a tip adapted to puncture the skin and/or are adapted to access the lumen on its own, also called tipped or tapered end catheters 70, as shown in FIG. 5. The tipped catheters can be well suited for medical procedures in an emergency room or intensive care units where time of the medical procedure is of most importance. To this end, tubular medical device 24 can include just the tipped catheter 70 instead of assembly 39 shown in FIG. 2. As shown in the figures, a portion of catheter 70 is configured to receive needle 22 before the needle is to be inserted in a body lumen.

In FIG. 5, catheter 70 can include an elongated body 72 extending distally from a hub assembly 74, with the length of the body being suitable to reach the intended internal target site. The catheter body 72 can have a proximal end 76 and a distal end 78, with one or more lumens extending therethrough. Catheter body 72 can be made of medical grade polymers, such as polyurethane or silicone. Hub assembly 74 can include a first port 80 where proximal end 76 of catheter body 72 attaches thereto, and one or more second ports 82. A fluid coupling 84, such as a standard Luer lock type connector, can be attached to second port 82, in order to provide external access to the lumen of catheter 70. Extending laterally from hub assembly 74 are attachment wings 86 that can be used to attach the catheter to the patient.

Also known as “tipping down” to provide a smoother transition, a portion 88 of catheter 70 proximate distal end 78 can taper to a smaller outer cross-sectional area. The smaller cross-sectional area can be sized approximately to match the outer cross-sectional area of needle 22, smaller than the general outer cross-sectional area of catheter body 72, to facilitate insertion of the catheter body 72 into a body lumen. Distal tapered portion 88 can include side port 66 as described herein. To this end, the various features discussed above with the side port, specifically with reference to FIG. 3 and FIGS. 4A-4D, can be applied to catheter 70 as appreciated by one skilled in the art.

Although catheter 70 is shown to include a single fluid coupling 84 and a single lumen, the catheter may have more than one fluid coupling and more than one lumen. For example, though not shown, one or more tubular members may extend proximally from second port 82 of hub assembly 74. Hub assembly 74 can be a structure that provides a fluid transition from one or more discrete tubular members (e.g., 1, 2, 3, 4, 5 or more tubular members) at one end to typically a corresponding number of separate, noncommunicating lumens (e.g., 1, 2, 3, 4, 5, or more lumens) that are located within catheter body 72. Further, a clamping device may be disposed around each of tubular members, which is capable of shutting off fluid flow through the tubular members and preventing air aspiration due to inadvertent hub dislodgement.

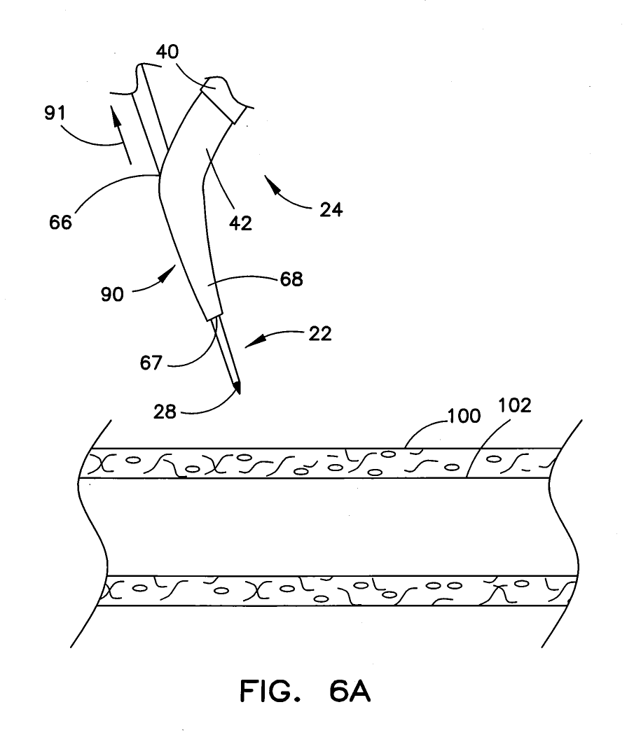

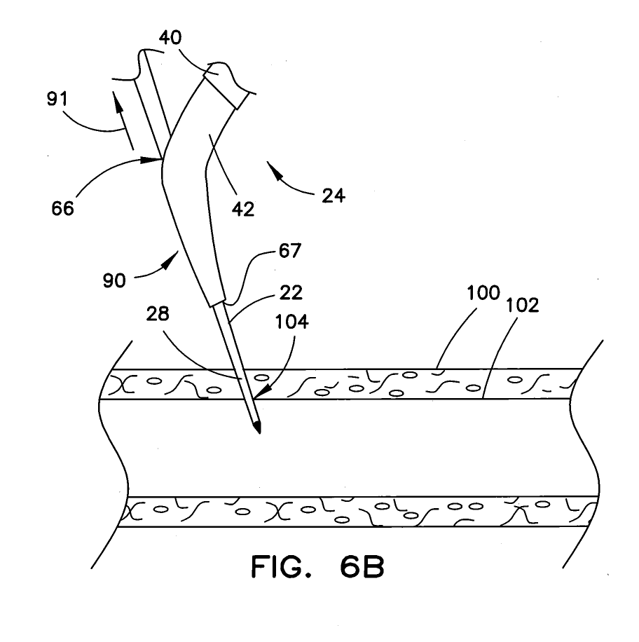

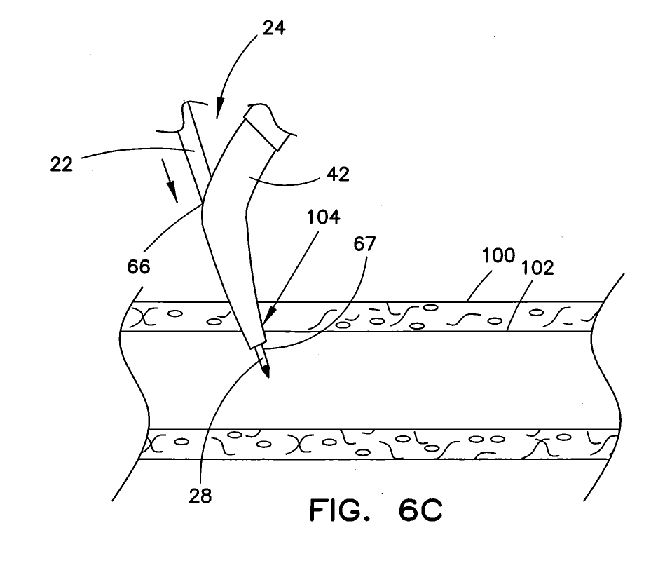

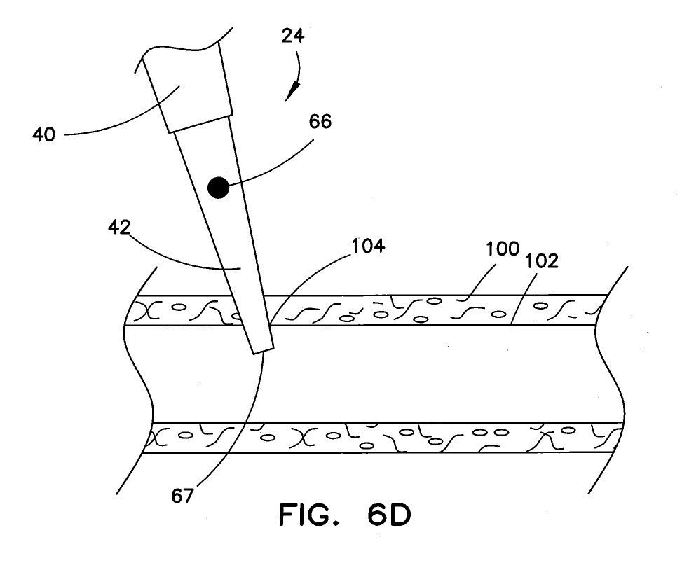

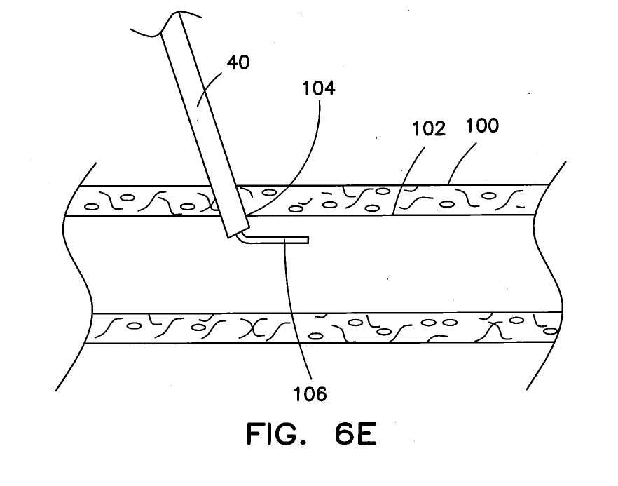

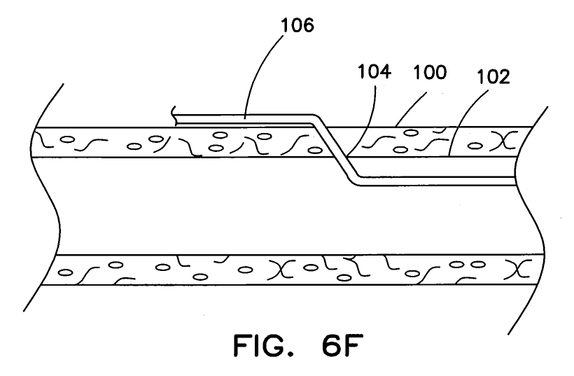

FIGS. 6A-6F illustrate a method for gaining percutaneous access to a body lumen with the devices such as needle 22 and one of the embodiments of tubular medical device 24, such as assembly 39 (FIG. 2) or catheter 70 (FIG. 5). Although the following method steps will focus on a tubular medical device that comprises assembly 39 having introducer sheath 40 and dilator 42 for later insertion of a non-tipped catheter, it can be appreciated by one skilled in the art that the following steps can also be applied when the tubular medical device comprises tipped catheter 70 instead.

A clinician typically gains percutaneous access by using a standard or modified Seldinger technique with a guidewire. The method described below is a Seldinger technique that is further modified to avoid the complications associated with guidewire, such as guidewire insertion within a needle.

First, as typically done, an access site is selected and prepped for needle insertion. The use of ultrasound may be helpful to determine the suitability of lumen access and patency, as well as the depth of the body lumen from the skin. According to FIG. 6A, distal tip 28 of needle 22 can be then inserted through side port 66 of tubular medical device 24, extended along a portion of lumen 68, and exited out distal end opening 67 of tubular medical device 24. It can be further appreciated that distal tip 28 of needle 22 can puncture the side wall of a tubular medical device without a side port already formed therein, to form side port 66 at the bedside of a patient. A distal portion 90 of tubular medical device 24 is now surrounding a corresponding portion of needle 22. Preferably, distal portion 90 is sufficiently flexible to bend at an angle that is at least oblique with respect to the general axis of the needle. Distal portion 90 can be then translated along needle 22 away from needle tip 28, in a direction represented by arrow 91, to leave an exposed portion of the needle. The exposed portion of the needle is typically greater than distance D so that the exposed portion can penetrate the body and body vessel, and so that when distal portion 90 is inserted into body, the side port 66 remains outside the body.

According to FIG. 6B, the exposed portion of needle 22 can then be inserted at the access site through skin 100 of a patient to form an entry opening 104 into a body lumen 102 of the patient, where external access to inside of lumen 102 is gained. Preferably, needle 22 can be inserted into skin 100 and body lumen 102 at an oblique angle in the range of about 45-60 degrees relative to an axis perpendicular to the body lumen. When a hollow needle is used, the clinician receives immediate feedback from blood flashback through the lumen of the needle to indicate a successful percutaneous access gained by the needle.

According to FIG. 6C, while needle 22 is in place within body lumen 102, distal portion 90 of tubular medical device 24 can be moved over needle 22 toward needle tip 28 so that distal end 67 of tubular medical device 24 can be inserted into entry opening 104. The outer cross-sectional area of distal end 67 is preferably about the same as the outer cross-sectional area of needle 22, and tapers to a larger outer cross-sectional area in the proximal direction. This taper arrangement can facilitate the entry of distal end 67 into entry opening 104, with the taper gradually widening and stretching entry opening 104 to a size for receiving a larger cross-sectional portion of tubular medical device 24. Tubular medical device 24 may need to be twisted in order to be advanced farther into lumen 102 to implant a portion of the tubular medical device within the subcutaneous portion of skin 100.

As a result, tubular medical device 24 can be inserted into the body lumen without the use of a conventional guidewire. This procedure avoids the complications associated with guidewire insertion within a needle, namely the skillful insertion of the guidewire through the needle, the risk of the needle popping out of the lumen during guidewire insertion, and the risk of needle occlusion from blood flashback and clotting. In an emergency or intensive care setting especially, the total time to gain percutaneous access for implantation of an interventional medical device is reduced as the procedure can be done in fewer steps since the guidewire is no longer required for insertion of the tubular medical device. The advantageous use of smaller gauge needles (less than 18-gauge needles) is now possible in order to minimize damage tissue or organs excessive bleeding that can be caused by larger gauge needles. For example, a 21-gauge thin wall needle generally having a 0.032 inch (0.81 mm) outer diameter and a 0.022 inch (0.56 mm) inner diameter can be used. Needles of 21-gauge or smaller are typically small enough to avoid damage to tissue or organs, or to cause excessive bleeding when inserted off target. Another benefit to smaller gauge needles is that the needles generally have corresponding shorter bevels at the needle tip as compared to the size of the bevel tip of an 18-gauge needle. Thus, it is much easier to get a shorter bevel into the lumen of a small lumen than the longer bevel of the 18-gauge needle.

Once a sufficient portion of the tubular medical device is inserted into body lumen 102, needle 22 can be removed from the body lumen. As to how much of the tubular medical device is inserted before removing the needle, the clinician can consider the placement site of the needle, anatomy of the patient, preference of the clinician, or the like, but is typically at least 3 cm. To remove the needle, the position of dilator 42 can be held inside lumen 102 so not to lose access through lumen entry opening 104, and the needle 22 is completely withdrawn from the tubular medical device, as shown in FIG. 6D. Tubular medical device 24 can then be straightened into a position so that more of the tubular medical device, such as the introducer sheath 40, can be translated within the lumen. Preferably, the tubular medical device can then be angled relative to the lumen at a more oblique angle in the range of about 60-75 degrees or less relative to an axis perpendicular to the body lumen. After the introducer sheath 40 has gained access to lumen 102, dilator 42 can then be removed by leaving introducer sheath 40 in place and unlocking the connector of the dilator from the handle of the introducer sheath. According to FIG. 6E, a non-tipped intravascular catheter 106 can then be inserted through introducer sheath 40. With introducer sheath 40 left in place, catheter 106 can be translated distally until the distal tip of the catheter is at a target site. However, it should be noted that a guidewire may be inserted within the dilator or the introducer sheath after percutaneous access has been gained, prior to insertion of the catheter if desired. After translating the guidewire to the target site, the catheter may then be inserted over the proximal end of the guidewire and then translated over the guidewire to the target site, after which the guidewire is removed from the body.

Introducer sheath 40 may be removed from around the catheter in order to leave the catheter in place for treatment or diagnostics, as shown in FIG. 6F. It can be appreciated by one skilled in the art that the introducer sheath can be removed from the catheter at any time so long as the catheter has gained sufficient access to the lumen. That is, removal of the introducer sheath can occur at any time including immediately right after the catheter gains sufficient access or even after the catheter has reached the target site. Before removal of introducer sheath 40, the location of the distal tip of catheter 106 can be radiographically verified with imaging equipment to ensure the distal tip is at its intended location. To remove a splittable introducer sheath such as sheath 40 that is implanted within the subcutaneous portion of the skin, the tabs of the handle can be pulled radially apart from one another and downward to snap or break the handle along the weakened region. After stabilizing catheter 106 in place within the body lumen 102, introducer sheath 40 can then be split apart by continuously pulling the tabs radially apart until the introducer sheath is entirely removed from catheter 106. This can be generally performed without affecting the position of catheter 106 within body lumen 102. A non-splittable sheath can be removed over the proximal end of the catheter.

The indication of a successful percutaneous access with the tubular medical device, such as the dilator or the introducer sheath, and/or the catheter can be monitored by coupling a syringe to a fluid coupling of the component and aspirating blood from the body lumen. When blood has aspirated, this is a positive indicator that the component still has percutaneous access to the body lumen. When no blood has aspirated, this indicates that the component does not have percutaneous access, and the steps should be followed to ensure successful percutaneous access. After a positive indicator, a second syringe containing sterile saline can be coupled to the fluid coupling of the component and used to flush the component.

The clinician can utilize the catheter in a variety of ways for treatment or diagnostics of the body lumen. As can be appreciated by one skilled in the art, one example of utilization can be delivery of a therapeutic, nutritional, and/or imageable agent. To this end, a syringe or an intravenous (IV) bag, can be coupled to a fluid connector of the catheter. The agent is pressurized in a manner to travel through the catheter and to be released to the target site. The catheter can provide short-term or long-term lumen pressure monitoring, blood sampling, administration of drugs and fluids, such as total parenteral nutrition (TPN), chemotherapeutic agents or other therapeutic drugs, and delivery of contrast in computed tomography (CT) studies with power injectors as known in the art.

The use of ultrasound may be helpful to determine the suitability of lumen access and patency. Ultrasound utilizes transducers, often called probes, which both generate and receive high-energy sound waves (ultrasound) with the use of quartz crystals by utilizing a principle called the piezoelectric effect. When the crystals receive an electric current, the crystals change shape and produce high-energy sound waves that travel outward toward the lumen, from external the body. Conversely, when sound or pressure waves bounce off the internal lumen (make echoes), the waves hit the crystals, which then emit electrical current. Therefore, the same crystals can be used to send and receive sound waves. A central processing unit processes the electrical currents emitted by the crystals as a result of the echoes, and the echo patterns are shown on a screen of an ultrasound machine to form a computer picture of body lumen called a sonogram or to indicate, for example, the position and depth of the lumen relative to the skin.

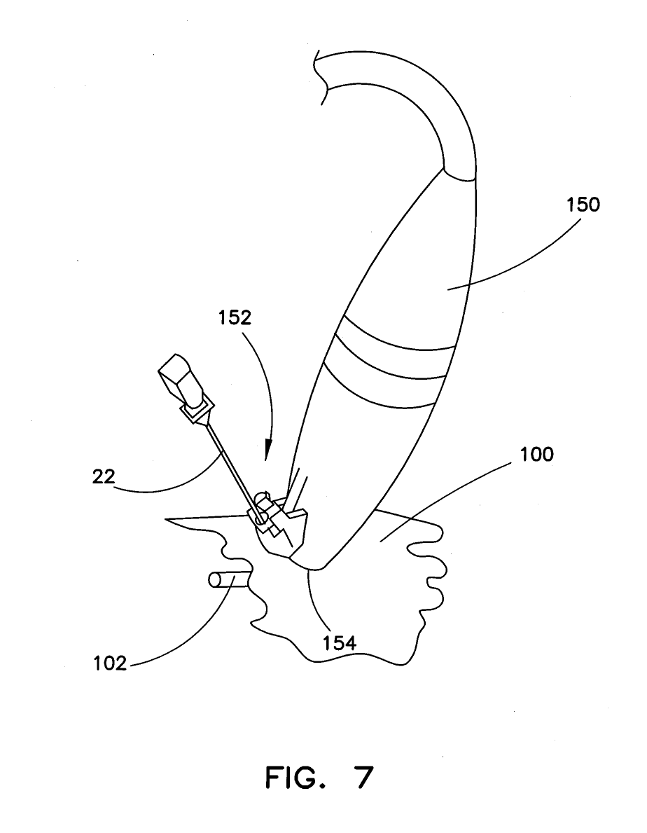

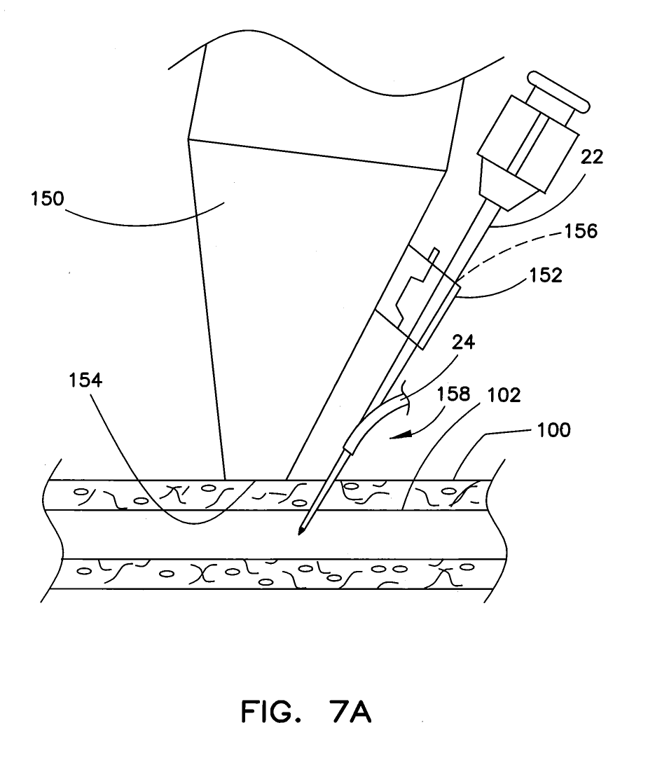

FIGS. 7-7A illustrate the use of an ultrasonic transducer probe 150. Probe 150 can include a needle guide holder 152 coupled along probe 150 toward a distal end 154 of probe 150. One such needle guide holder, as well as an ultrasound needle guide kit, is available from Sheathing Technologies, Inc. (Morgan Hill, Cal.). Needle guide holder 152 is adapted to orient and stabilize needle 22 at a suitable angle relative to the body lumen 102, such as an angle in the range of about 45 to 60 degrees relative to an axis perpendicular to the body lumen. Needle guide holder 152 can include a receiving lumen 156 extending therethrough that is sized to receive the needle. It is preferable that a portion of the needle guide holder is capable of frictionally engaging the needle to hold it in place. As can be seen, needle guide holder 152 should be located a certain distance from skin engaging end 154 of probe 150 so that a portion 158 of tubular medical device 24 can fit between needle guide holder 152 and skin 100 while the distal tip of needle 22 is in body lumen 102. This arrangement can facilitate the insertion of the needle into the body lumen, and may facilitate the insertion of the tubular medical device over the needle into the body lumen.

The system and method can facilitate rapid percutaneous access to the body lumen preferably without the use of a guidewire. To this end, complications associated with guidewire use, such as inadvertent “pop out” of needle and occlusion of the needle lumen during guidewire manipulation and insertion can be avoided. A smaller needle (such as 21-gauge or smaller) can be used since it no longer needs to be sized to accommodate such a large guidewire that is often required to provide sufficient mechanical support and guidance for interventional medical devices. In addition, percutaneous access to a body lumen can be gained in fewer steps, and with such rapidity that the system and method may be ideal for emergency room settings. The side port can be formed in the tubular medical device at a manufacturing facility under a controlled environment suitably acceptable for production of medical devices, or can be formed with the puncture of the needle distal tip through the side wall of the tubular medical device at the bedside of a patient. Other advantages will become readily apparent from the examples described herein.

Drawings in the figures illustrating various embodiments are not necessarily to scale. Some drawings may have certain details magnified for emphasis, and any different numbers or proportions of parts should not be read as limiting, unless so designated in the present disclosure. Those skilled in the art will appreciate that embodiments not expressly illustrated herein may be practiced within the scope of the present invention, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

1. A system for gaining percutaneous access to a body lumen, comprising:
a needle having a distal tip configured to create an entry opening in said body lumen; and
a tubular medical device having a proximal end, a distal end opening, a proximal portion, and a distal portion, the distal portion having a side port formed in a wall thereof and the proximal portion extending proximally from the side port, the side port being in communication with the distal end opening through a passageway extending at least between the side port and the distal end opening, and the proximal portion and the distal portion forming a smooth contiguous outer surface of the tubular medical device,
wherein the side port, the passageway, and the distal end opening are sized to receive said needle so that the distal tip of the needle is extendable therethrough beyond the distal end opening of the tubular medical device and the proximal portion is angled away from the axis of the needle before said entry opening is formed in said body lumen, an exterior opening of the side port being smooth with the contiguous outer surface of the tubular medical device and not extending outward from the outer surface thereof.

2. The system of claim 1, where the needle further comprises a lumen extending therethrough, and the needle distal tip has an opening, the needle lumen being in communication with the opening at the needle distal tip.

3. The system of claim 1, where the needle further comprises a lubricious coating along a length of an exterior surface of the needle.

4. The system of claim 1, where the tubular medical device comprises an assembly of an introducer sheath and a dilator inserted within the introducer sheath, where the dilator comprises said, side port, said proximal portion, and said distal portion, and said side port is positioned proximate a distal end of the introducer sheath.

5. The system of claim 1, where an inner surface defining said passageway of said tubular medical device comprises one or more annular protrusions.

6. The system claim 1, where at least a length of an inner surface defining said passageway comprises a lubricious coating.

7. The system of claim 1, where the tubular medical device comprises a catheter including said side port, said proximal portion, and said distal portion.

8. The system of claim 7, where said catheter further comprises a catheter body having one or more lumens extending longitudinally therethrough, a hub assembly and at least one tube, the catheter body extending distally from the hub assembly, the at least one tube having a lumen extending longitudinally therethrough, and extending proximally from the hub assembly, and the hub assembly having a body configured to provide a fluid transition from the lumen of the at least one tube to the one or more lumens of the catheter body.

9. A system for gaining percutaneous access to a body lumen through an access opening without a guidewire, comprising:
a tubular medical device having a proximal end and a distal end opening, a proximal portion, a tapered distal portion to dilate said access opening of said body lumen, and a side port formed through a wall of the tapered distal portion, the proximal portion extending proximally from the side port, the side port being in communication with the distal end opening through a passageway extending therebetween, and the proximal portion and the distal portion forming a smooth contiguous outer surface of the tubular medical device,
a needle having a distal tip configured to create said access opening in said body lumen, where the needle resides within the side port, the passageway, and the distal end opening so that the distal tip is extended beyond the distal end opening of the tubular medical device and the proximal portion is angled away from the axis of the needle for creation of said access opening, an exterior opening of the side port being smooth with the contiguous outer surface of the tubular medical device and not extending outward from the outer surface thereof,
where a portion of the tapered distal portion is translatable over the needle into said access opening, and said needle is removable from the side port, the passageway, and the distal end opening.

10. The system of claim 9, wherein the side port of the tubular medical device is sized approximately the same as an outer cross-sectional area of said needle.

11. The system of claim 9, wherein the tubular medical device comprises an assembly of an introducer sheath and a dilator inserted within the introducer sheath, where the dilator comprises said side port, said proximal portion, and said tapered distal portion, and said side port is positioned proximate a distal end of the introducer sheath.

12. The system of claim 9, where a length of at least one of an outer surface of the needle and an inner surface defining the passageway includes a lubricious coating.

13. The system of claim 9, where an inner surface defining the passageway includes one or more annular protrusions.

14. The system claim 9, where the tubular medical device comprises a catheter including said side port, said proximal portion, and said tapered distal portion.

15. A method for gaining percutaneous access to a body lumen, the method comprising the steps of:
providing a tubular medical device having a proximal end, a distal end opening, a proximal portion, a distal portion, a side port formed in the distal portion and the proximal portion extending proximally from the side port, and a passageway in communication with at least the distal opening end and the side port, and the proximal portion and the distal portion forming a smooth contiguous outer surface of the tubular medical device;
providing a needle having a distal tip configured to create an entry opening in said body lumen;
extending the distal tip of said needle through the side port and translating the needle distal tip along the passageway to exit beyond the distal end opening of the tubular medical device and the proximal portion is angled away from the axis of the needle, an exterior opening of the side port being smooth with the contiguous outer surface of the tubular medical device and not extending outward from the outer surface thereof;
forming an entry opening in said body lumen with the distal tip of the needle, while the needle resides within the side port, passageway, and the distal end opening; and
advancing the distal portion of the tubular medical device over the indwelling needle into the entry opening so that the tubular medical device gains percutaneous access to the body lumen.

16. The method of claim 15, further comprising the step of removing the needle from the side port of the tubular medical device, while maintaining a portion of the tubular medical device in place within the body lumen.

17. The method of claim 15, further comprising retaining the needle in a fixed angular position relative to the body lumen with a holder of an ultrasonic probe prior to the forming an entry opening step, and translating the needle through the holder to form the entry opening in said body lumen with the distal tip of the needle, while the needle resides within the side port, passageway, and the distal end opening.

18. The method of claim 15, wherein the tubular medical device comprises an assembly of an introducer sheath and a dilator inserted within the introducer sheath, where the dilator comprises said side port, said proximal portion, and said distal portion, and said side port is positioned proximate a distal end of the introducer sheath.

19. The method of claim 15, wherein the tubular medical device comprises a catheter including a catheter body having one or more lumens extending longitudinally therethrough, a hub assembly and at least one tube, the catheter body extending distally from the hub assembly, wherein the catheter body includes the said side port, the proximal portion, and said distal portion, the at least one tube having a lumen extending longitudinally therethrough, and extending proximally from the hub assembly, and the hub assembly having a body configured to provide a fluid transition from the lumen of the at least one tube to the one or more lumens of the catheter body.

20. The method of claim 15, where the advancing step is performed without using a guidewire.
